(19) United States
(12) Patent Application Publication
(10) Pub. No.: US 2019/0224354 A1
(43) Pub. Date: Jul. 25, 2019

(54) COLD PLASMA DEVICES FOR DECONTAMINATION OF FOODBORNE HUMAN PATHOGENS

(71) Applicant: THE BOARD OF REGENTS FOR OKLAHOMA STATE UNIVERSITY, STILLWATER, OK (US)

(72) Inventors: Li Maria MA, STILLWATER, OK (US); Kedar K. PAI, Stillwater, OK (US); Jamey D. JACOB, Stillwater, OK (US); Chris TIMMONS, STILLWATER, OK (US)

(21) Appl. No.: 15/738,731

(22) PCT Filed: Jul. 25, 2016
(86) PCT No.: PCT/US2016/043899

§ 371 Date: Dec. 21, 2017

Related U.S. Application Data
(60) Provisional application No. 62/196,769, filed on Jul. 24, 2015.

(51) Int. Cl. A61L 2/14; H05H 1/24

(52) U.S. Cl. CPC A61L 2/14; H05H 1/2406; Y10T 29/49147; H05H 2001/2412; H05H 2001/2425; H05H 2001/2418

(57) ABSTRACT

Methods and systems for decontaminating food products includes arranging a first electrode and second electrode in an asymmetric relationship on opposite sides of a dielectric layer, providing an insulating covering on the first electrode, and applying a power source to the first and second electrodes. A voltage is applied between the first electrode and the second electrode in ambient atmosphere to create a cold plasma and a food product is decontaminated by the plasma.

### CROSS REFERENCE TO RELATED APPLICATIONS

This application claims the benefit of U.S. Provisional Patent Application Ser. No. 62/196,769 filed on Jul. 24, 2015, and incorporates said provisional application by reference into this document as if fully set out at this point.

### TECHNICAL FIELD

This disclosure relates generally to systems and methods for decontaminating food products and, more specifically, to systems and methods of using cold plasma devices to decontaminate same.

### BACKGROUND

Due to the increasing demand for locally grown produce, supermarkets and other food retailers have pledged to reduce food miles (miles from source to point of sale) and increase its purchase of “local” produce. The numbers of medium- to small-scale producers are currently rising exponentially. At the same time, partly because of public education and broad media coverage on foodborne illness outbreaks, more and more consumers have become aware of food safety issues. Both groups are constantly looking for affordable and safer ways to control their food safety; however, currently there are very few service providers catering to this market.

What is needed is a system and method for addressing the above, and related, concerns.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

The invention of the present disclosure, in one aspect thereof, comprises a method including arranging a first electrode and second electrode in an asymmetric relationship on opposite sides of a dielectric layer, providing an insulating covering on the first electrode, and applying a power source to the first and second electrodes. A voltage is applied between the first electrode and the second electrode in ambient atmosphere to create a cold plasma and a food product is decontaminated by the plasma.

In some embodiments, the insulating covering is arranged to create an enclosure. The enclosure may comprise a cylinder. The first electrode and second electrode may be arranged in the cylinder to promote the flow of air produced by the plasma through the cylinder. The food product may be a powder flowing through the cylinder.

In some embodiments, the dielectric and the insulated covering form a grid defining a plurality of perforations therethrough. The first electrode and second electrode may be arranged to promote flow of gases through the grid. Again, the food product may be placed in the grid. In some embodiments the dielectric layer may be placed in proximity to a food carrying conveyor system for decontamination of food items in transit on the conveyor system. In other embodiments, the dielectric layer and the insulating covering may be formed into a portion of a container for decontamination of contents of the container.

The invention of the present disclosure, in another aspect thereof, comprises a method including placing a substrate so as to define at least a portion of an interior volume, placing a dielectric layer on the substrate in the interior volume, and placing a plurality of electrodes immediately adjacent to the dielectric layer such that at least one electrode is exposed to the interior volume and at least one electrode is insulated by the substrate. A food product may be placed into the interior volume in the presence of ambient atmospheric gases, and an excitation voltage provided between the electrodes to produce cold plasma directed to contact with the food product.

The plurality of electrodes may comprise at least two electrodes in a symmetric relationship with respect to one another on opposite sides of the dielectric. The step of placing a plurality of electrodes may further comprise placing at least two electrodes in an offset relationship with respect to one another on opposite sides of the dielectric. Plasma may be produced for contact with the food product long enough to destroy food borne pathogens.

In some embodiments, a voltage is applied to electrodes on both sides of the dielectric layer. The substrate may be formed into a cylinder such that plasma is produced inside the cylinder. In such cases, the substrate may be arranged into multiple cylinders, each with a plurality of electrodes such that plasma is produced inside each cylinder. Interior electrodes may also be arranged in a spiral within the cylinder. A three dimensional chevron shape may also be formed from the substrate such that plasma is produced therein.

### BRIEF DESCRIPTION OF THE DRAWINGS

These and further aspects of the invention are described in detail in the following examples and accompanying drawings.

FIG. 1 is a schematic diagram of one embodiment of a plasma generating device according to the present disclosure.

FIG. 2 is a schematic diagram of another plasma generating device according to the present disclosure.

FIG. 3 is a schematic diagram of a plasma decontamination system according to the present disclosure.

FIG. 4 is a side profile view of some example relative positions of upper and lower conductors that are suitable for use with various embodiments the present disclosure.

FIG. 5 provides schematic illustrations of linear and annular example electrode configurations of the present disclosure.

FIG. 6 is a plan view of an annular embodiment of an electrode configuration.

FIG. 7 is a side profile view of a progression of relative motive force for some configurations of the embodiment of FIG. 6.

FIG. 8 is a side profile view of a progression of asymmetrical motive force that may be produced by the embodiment of FIG. 6.

FIG. 9 is a plan view of another embodiment of the present disclosure employing multiple annular electrodes.

FIG. 10 is a cross sectional view of another annular embodiment of the present disclosure.

FIG. 11 contains schematic illustrations of additional electrode configurations of the present disclosure.

FIG. 12 is a perspective view of a plasma pouch decontamination device according to the present disclosure.

FIG. 13 is an end cutaway view of the plasma pouch of FIG. 12.

FIG. 14 is a perspective view of a system employing the plasma pouch of FIG. 12 for decontamination purposes.

FIG. 15 illustrates an embodiment of a surface dielectric barrier discharge (SDBD) symmetric electrodes arrangement.

FIG. 16 is a schematic diagram of a system for cold plasma treatment of bacterial foodborne pathogens according to the present disclosure.

FIG. 17 is a plotted comparison of inactivation of a 5 strain cocktail of Listeria monocytogenes pathogen on glass coverslips using asymmetric and symmetric electrode arrangement SDBD actuators and placing the actuator at various heights in the system of FIG. 16.

FIG. 18 is a data table of average D-values for Salmonella, STEC, and Listeria after 2 min of cold plasma treatment at 1, 3, 5, and 7 cm in the system of FIG. 16.

FIG. 19 contains plots of average log reductions in bacterial populations after 2 and 4 min treatments with cold plasma at 1, 3, 5, and 7 cm in the system of FIG. 16.

FIG. 20 is perspective view of a cold plasma system having a grid-like substrate.

FIG. 21(a) is a schematic diagram of a cold plasma compact system having a 3-dimensional chevron shape.

FIG. 21(b) is a perspective view of the system of FIG. 21(a).

FIG. 22 is a simplified schematic diagram of a semi-cylindrical electrode configuration.

FIG. 23 is a simplified schematic cutaway diagram of a cylindrical electrode configuration.

FIG. 24 is a ghost view of the cylindrical electrode configuration of FIG. 23 formed into a plasma generation device with induced flow along the cylinder.

FIG. 25 is a perspective view of a cold plasma system built upon an array of cylindrical electrodes.

FIG. 26 is a schematic view of a cold plasma generation system for decontaminating items on a conveyor system.

FIG. 27 is a schematic view of a container integrating a cold plasma generation system.

### DETAILED DESCRIPTION

As a relatively new microbial inactivation technology, nonthermal or cold plasma has been gaining a lot of interest in applications related to food safety. Various modes of plasma generation have been explored. However, these designs require high power input and an artificial gas flow, complicating their practical applications. Further, the prior art approaches to inactivating food pathogens have utilized a noble (inert) gas—instead of atmospheric gas—as a means of generating plasma, the disadvantages of which should be clear.

Various embodiments of the present disclosure provide systems and method for inactivating food-borne pathogens. Various embodiments of the present disclosure utilize cold plasma generated from atmospheric or ambient gas. As discussed in detail below, devices of the present disclosure are surface dielectric barrier discharge (SDBD) cold plasma devices that may be constructed with electrodes placed asymmetrically or symmetrically around the dielectric material. Atmospheric cold plasma offers a dry, non-thermal, and rapid process for decontamination of food products, and food contact surfaces, among other items. Food products, for purposes of the present disclosure, refer to items intended for human or animal consumption and which might be susceptible to microbiological contamination. These food products may be raw, precooked, or processed and may be ready to eat or may include constituent ingredients for recipes, or some stage in between. Microbiological contaminants are defined as bacteria, virus, fungi, and protozoa or their toxins and by-products present in food or on contact food surfaces. Microbiological contaminants are destroyed or denatured by exposure to plasma generated by the systems and methods of the present disclosure. It should be understood that food or food substances of any physical form or shape may be treatable with the systems and methods of the present disclosure. For example, cuts of meat, fruits, or vegetables, or more processed and/or irregularly shaped food products are suitable for decontamination according to the present disclosure. Nuts, grains, legumes, flours, powders, pellets, and other forms are also suitable for decontamination according to systems and methods of the present disclosure. It will be appreciated from the specific descriptions of the various embodiments of the present disclosure that the disclosed SDBD systems can both generate cold plasma from ambient atmosphere and propel it to contaminated locations upon irregularly shaped food products in sufficient quantities to provide meaningful and substantial decontamination or disinfection.

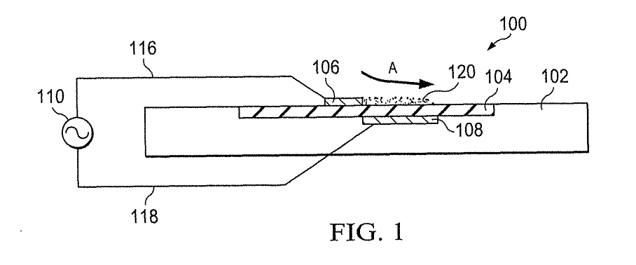

Referring now to FIG. 1, a schematic diagram of one embodiment of a plasma generating device according to the present disclosure is shown. In the embodiment of FIG. 1, the device 100 includes a substrate 102 onto which the various other components described herein may be attached. As will be explained in greater detail below, the substrate 102 could be a portion of a chamber or enclosure. A suitable substrate 102 would be a non-conductive, impermeable material that is resistant to high temperatures or gas species. Glass, acrylic or phenolic materials are examples of acceptable materials.

Integrated with the substrate 102, or forming a part of the substrate 102, is a dielectric layer 104. The dielectric layer 104 could be formed, by way of example only, from any material with a low dielectric constant such as PTFE, kapton, or ceramic.

An electrode 106 is situated along a top surface of the dielectric layer 104. A second electrode 108 is situated along a lower surface of the dielectric layer 104. It can be seen that the electrodes 106, 108, are at least somewhat offset from one another along a length of the dielectric layer 104. The electrodes 106 and 108 might be made of copper or any other material with suitable conductivity.

The electrode 106 attaches to a voltage source 110 by an electrical lead 116. The electrode 108 attaches to the voltage source 110 by an electrical lead 118. In the present embodiment, the voltage source 110 may include a power supply as well as any necessary transformers or circuit conditioning components to enable generation of plasma by application of sufficient voltage between the electrodes 106, 108 on the surface of the dielectric layer 104. In the present embodiment, a plasma region 120 develops between the first electrode 106 and the second electrode 108. The plasma region 120 also provides a motive force for any adjacent gases in the direction of the arrow “A”.

Various duty cycles and voltages may be utilized to generate plasma. In the present embodiment, various voltages, frequencies and duty cycles have been tested and found to be operational. By way of example only, these include voltages in the range of 5 to 50 kV at frequencies of 1,000 to 10,000 Hz at a 10% to 100% duty cycle at modulated frequencies of 1, 2, 5, 10, 100, 500 and 5000 Hz. It will be appreciated that various flow rates and associated decontamination characteristics can be generated by adjusting the duty cycle voltage and frequency of the applied voltage. In application, the limit is most likely to be the durability of the materials used to construct the device 100 and the available power supply. For example, if operating from commercial power, higher voltages may be available than if operating from battery power.

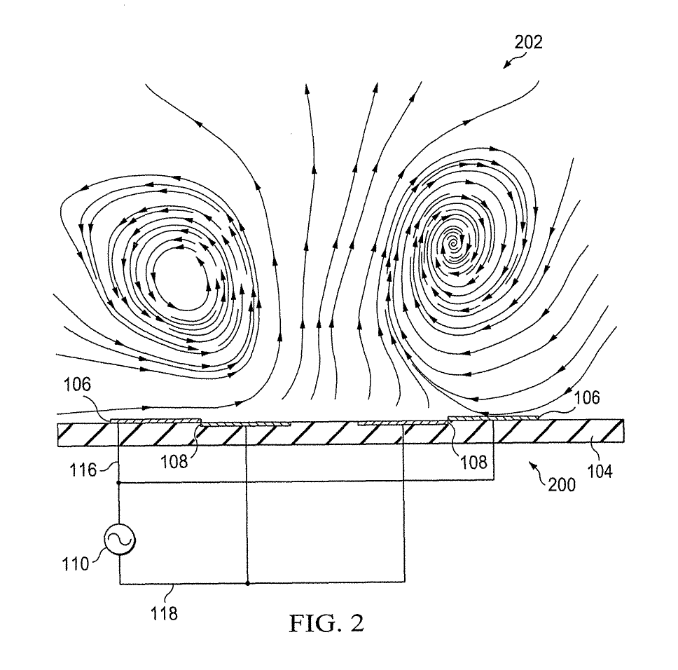

Referring now to FIG. 2, a schematic diagram of another plasma generating device according to the present disclosure is shown. The device 200 is similar in construction and operation to the device 100 of FIG. 1. In the present device, two upper electrodes 106 are attached opposite a dielectric layer 104, and are offset from a pair of lower electrodes 108. Electrical lead 116 attaches the upper electrodes 106 to the voltage source 110 and a lower electrical lead 118 attaches the lower electrodes 108 to the voltage source 110.

In the present embodiment, it will be appreciated that, due to the configuration of the electrodes 106 relative to the electrodes 108, flow regions that are pointed in substantially opposite directions will be achieved. Thus, each electrode pair 106, 108, will generate plasma as well as a motive force pointed inward according to FIG. 2. This will cause a swirling effect of any adjacent gases as illustrated by the exemplary flow lines 202.

In FIG. 2, both of the upper electrodes 106 are shown attached to a common voltage line 116. Similarly, the lower electrodes 108 are shown attached to a common voltage line 118. Thus, in operation, in this embodiment the upper electrodes 106 will always be at the same voltage potential while the lower electrodes 108 will likewise share a voltage potential. However, it is understood that other configurations are possible. For example, both of the upper electrodes 106 need not necessarily be operated at the same voltage level. Similarly, the lower electrodes 108 could be attached to different voltage levels. In this manner the device 200 may be operated in a pulsing fashion where the gas flow is first in one direction, and then in another. It will be appreciated that both of the aforedescribed exemplary operating methods will result in a thorough mixing of gases next to and around the device 200. Thus, over time the adjacent gases will be exposed to the plasma generated by the device and the air thereby decontaminated from biological agents.

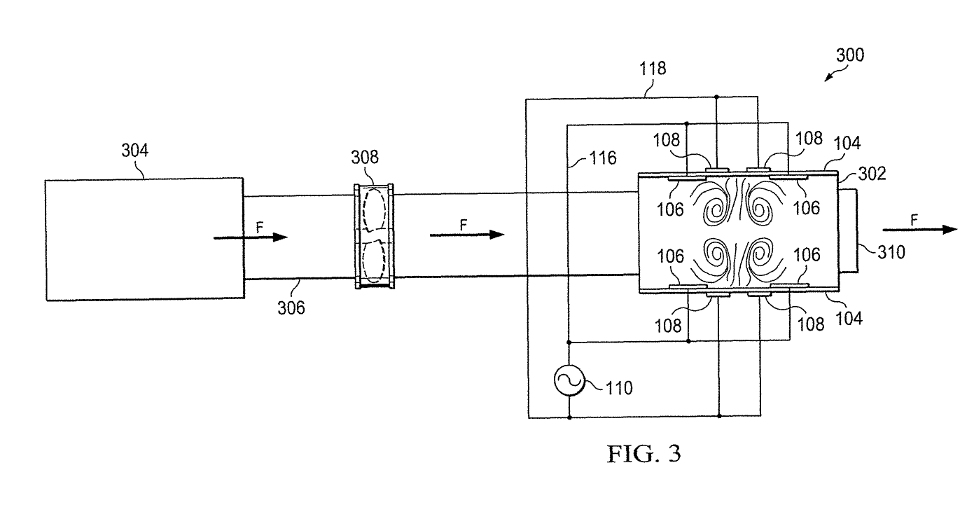

Referring now to FIG. 3, a schematic diagram of a plasma decontamination system according to the present disclosure is shown. The plasma decontamination system 300 comprises a plasma decontamination chamber 302. This chamber 302 may have a plurality of inner electrodes 106 separated from a plurality of outer electrodes 108 by a dielectric layer 104. The dielectric layer 104 may be enclosed by a substrate (not shown).

The inner electrodes 106 may attach to a voltage source 110 by a lead 116. The outer electrodes 108 may attach to the voltage source 110 by a lead 118. The plasma decontamination system 300 operates in a manner similar to those previously described in that voltages will be applied to the plurality of inner electrodes 106 and outer electrodes 108 generating plasma inside the plasma decontamination chamber 302. The motive forces provided by the plasma generation will serve to mix and swirl gas within the plasma decontamination chamber 302 such that the gases inside of the chamber 302 may be substantially completely decontaminated from biological agents.

In some embodiments, the motive force for drawing contaminated air into the plasma decontamination chamber 302, and expelling decontaminated air, will be entirely due to the location and configuration of the plasma generating electrodes 106, 108 in and on the plasma decontamination chamber 302. However, in other embodiments, a separate flow control system may be utilized that provides for selective introduction of contaminated gases into the decontamination chamber 302 from a contamination source 304. The contamination source 304 could be naturally or otherwise occurring bacteria or viruses, medical waste, sewage or any number of sources which generate air containing bio-contaminants. In the present embodiment, the gases flow generally from the contamination source 304 in the direction of the arrows “F”.

A conduit 306 is provided between the plasma decontamination chamber 302 and the contamination source 304. A fan 308 may be provided that produces vacuum toward the contamination source 304, and positive pressure toward the plasma decontamination chamber 302. The fan 308 or other flow driving device may operate in an open-loop configuration or may be selectively activated such that air within the decontamination chamber 302 has sufficient time for exposure to plasma to achieve a satisfactory level of decontamination. An exit conduit 310 may be provided for moving the decontaminated gas away from the decontamination chamber 302. In some embodiments, the exit conduit 310 will merely function as a selectively closeable valve to prevent air from escaping the decontamination chamber 302 until sufficiently and effectively decontaminated.

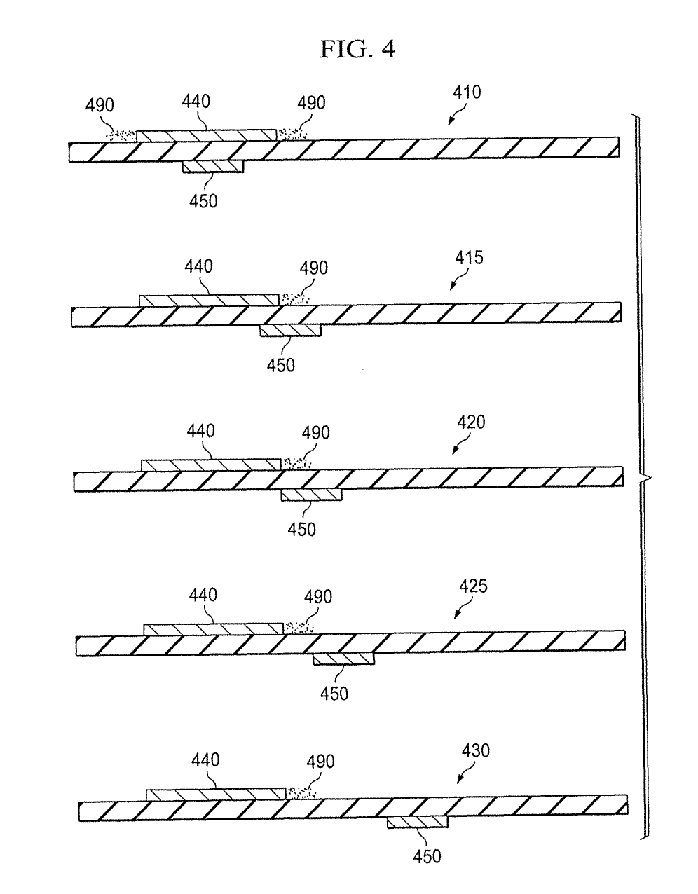

FIGS. 4 through 11 illustrate additional embodiments of the present disclosure. In FIG. 4, configuration 410 is an embodiment that operates to generate a plasma stream 490 on both sides of the upper conductor 440 at its periphery. However, some embodiments tend to produce better results when the upper 440 and lower 450 conductors at least partially overlap, tends to produce better results (e.g., 410 and 415). Further, and continuing with the examples of FIG. 4, configurations such as 420 to 430 tend to show generally decreasing performance as compared with configuration 415. Obviously, if the conductors are spaced sufficiently far apart the plasma generated will be negligible or zero.

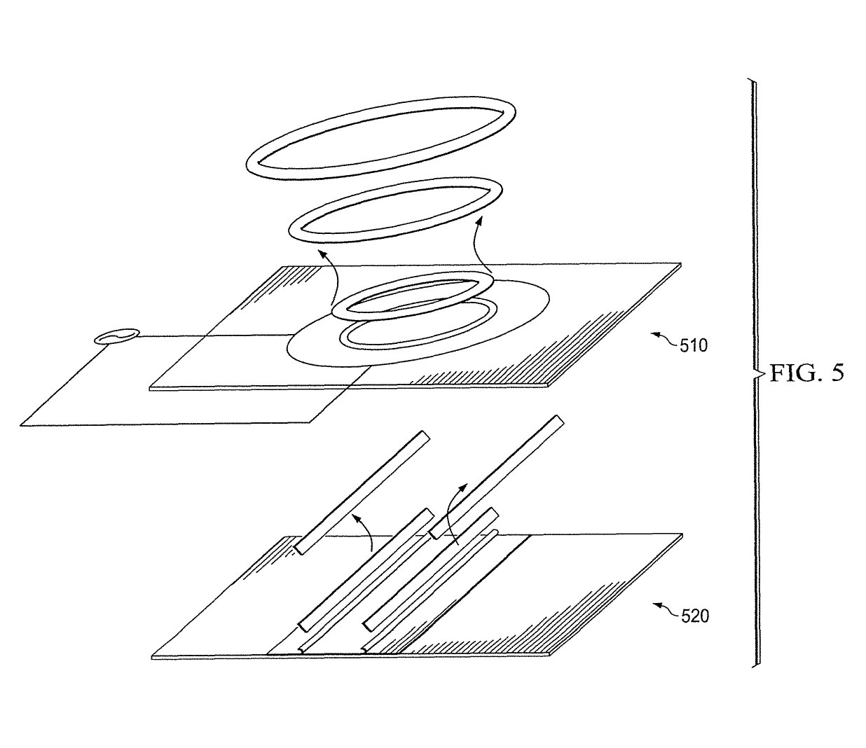

FIG. 5 contains a schematic illustration of linear 520 and annular 510 embodiments. As can be seen, in the embodiments of this figure the motive force associated with the plasma stream is in an outward (upward by reference to this figure) direction, i.e., a “blow” embodiment. That being said, if the electrical leads are reversed, a downward/inward (i.e., a “suck”) embodiment can be created.

FIGS. 6 and 7 contain additional details of an annual embodiment. In the configuration of FIG. 6, note that the amount of plasma generated and the corresponding motive force can be varied by increasing the voltage differential that is supplied to the electrodes 610 and 620 as is illustrated generally in FIG. 7.

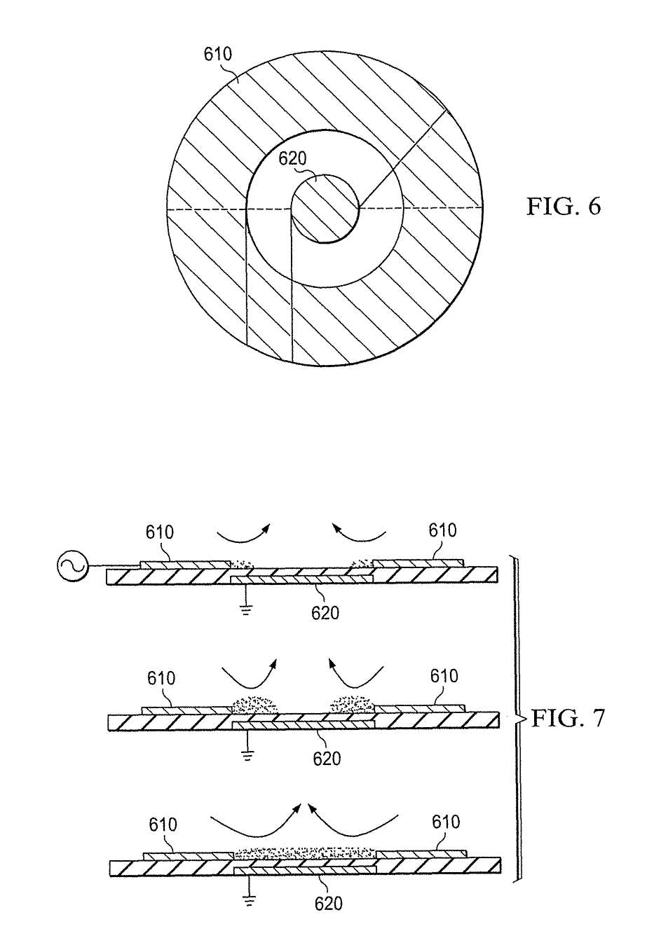

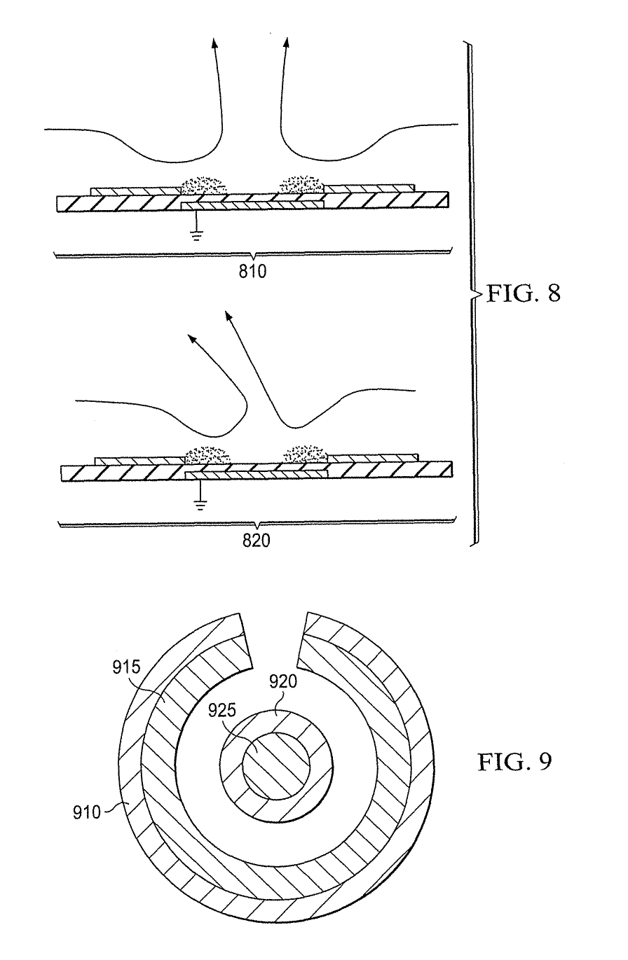

FIG. 8 is a schematic cross-sectional illustration of the embodiment of FIG. 7 that shows that, although the motive force is generally directed orthogonally away from (or toward) the dielectric material, in some configurations and at some points along the embodiment of FIG. 7, the force may take a path that is non-orthogonal to the dielectric material.

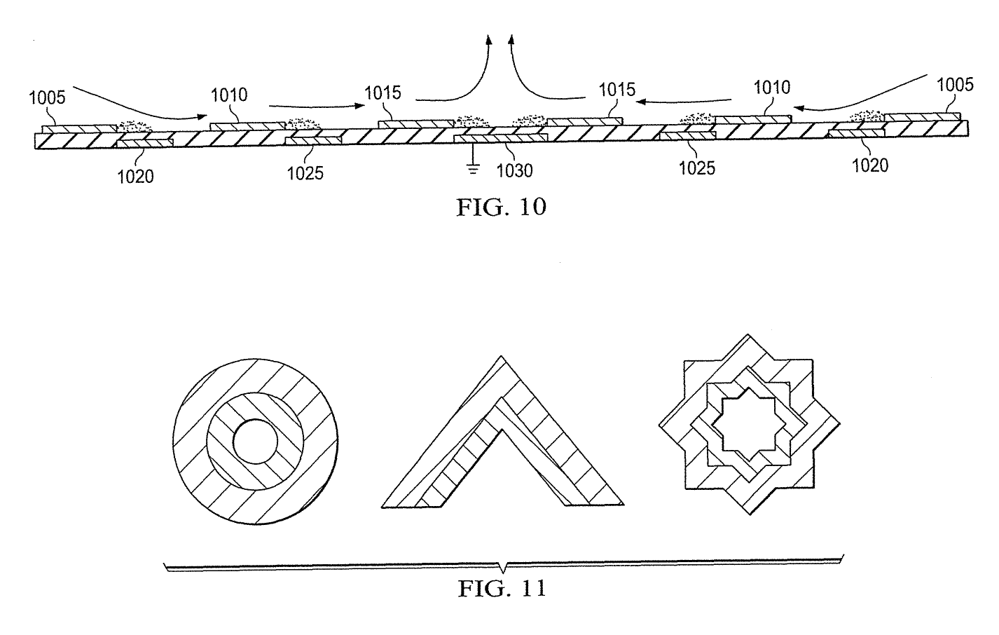

FIGS. 9 and 10 are schematic illustrations of still other arrangements that are generally annular. FIG. 9 contains an illustration of an annular embodiment that includes two upper electrodes 910 and 920 and two lower electrodes 915 and 925. Note that the electrodes 910 and 920 might be electrically isolated from each other or not. The same might also be said with respect to electrodes and 915 and 925.

FIG. 10 contains a cross-sectional view of still another annular embodiment, with upper electrodes 1005, 1010, and 1015, and lower electrodes 1020, 1025, and 1030. Note that in some embodiments (e.g., FIGS. 7, 8, and 10) one or more electrodes, e.g., the lower electrode in these figures, is embedded in the dielectric.

FIG. 11 contains some further embodiments, e.g., annular, chevron, and hybrid. Those of ordinary skill in the art will readily be able to devise other shapes and arrangements that generate plasma according to the instant disclosure.

Note that, although in some embodiments the dielectric is a generally rectangular single planar surface, in other embodiments it might be round, polygonal, etc. Additionally, in still other embodiments the dielectric might be separated into two or more pieces that are interconnected by conductive material. In such an instance, the electrodes of the instant disclosure might be placed on the same or different pieces of the dielectric. The dielectric and/or associated electrodes might also be non-planar depending on the requirements of a particular application. Thus, for purposes of the instant disclosure it should be understood that the term “dielectric” is applicable to materials that are any shape, that are planar or not, and that might be divided into multiple pieces that are joined by conductive materials.

Further note that for purposes of the instant disclosure, the term “length” should be broadly construed to be any linear dimension of an object. Thus, by way of example, circular dielectrics have an associated length (e.g., a diameter). The width of an object could correspond to a length, as could a diagonal or any other measurement of the dielectric. The shape of the instant electrodes and associated dielectric are arbitrary and might be any suitable shape.

Still further, note that the voltages applied to the top and bottom electrodes may be different. It is important that the voltage differential between the electrodes be sufficient for the generation of plasma, e.g., about 5 to 50 kV as was discussed previously. The positive electrode can either be on the top or the bottom of the dielectric and the orientation might be varied depending on the direction it is desired to have the plasma stream move.

Finally it should be noted that the term “offset” as used herein should be broadly construed to include cases where there is no overlap between the electrodes (e.g., configurations 425 and 430) as well as cases where there is substantial overlap (e.g., configuration 410). What is important is that the edges of the upper and lower electrodes not be completely coincident, e.g., one electrode or the other should have a free edge (or part of an edge) that does exactly overlay the corresponding electrode on the opposite surface.

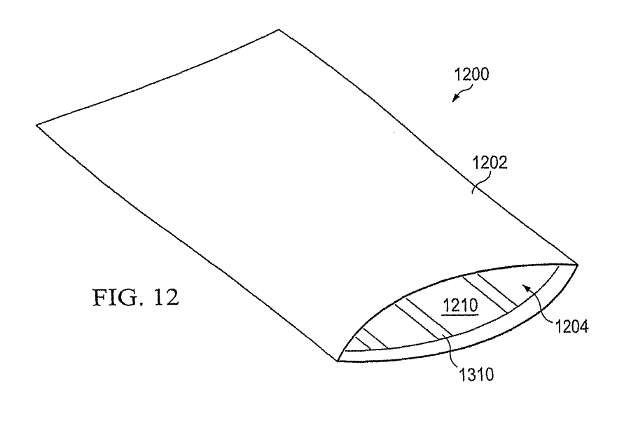

Referring now to FIG. 12 a perspective view of one embodiment of a plasma pouch decontamination device according to the present disclosure is shown. The pouch 1200 represents on application of the plasma generation devices disclosed herein. The pouch 1200 may be constructed in various sizes to allow sterilization of differently sized articles. For example, the pouch 1200 can have multiple compartments like a piano file, and/or it can be constructed to substantially conform to the geometric outline of the object device to be disinfected or sterilized. In other examples, the pouch 1200 can be produced as a mitten. A mitten or glove configuration may be constructed “inside out” such that plasma is generated on the exterior (e.g., for hand held decontamination of instruments). Some embodiments will provide a sheath-like sterilization pouch, which can be used to decontaminate the surfaces of long, serpentine bodies such as those of catheters and other devices.

The pouch 1200 may comprise a body portion 1202 that may be folded around on itself to create an interior 1210 of the pouch 1200. The body portion 1202 may be sealed at all but one edge that forms an opening 1204. The opening 1204 allows for insertion and removal of articles to be sterilized. Within the interior 1210 of the pouch 1200 a plurality of plasma-generating electrodes 1310 can be seen. These electrodes 1310 may cover a portion, or substantially all, of the interior 1210 of the pouch 1200.

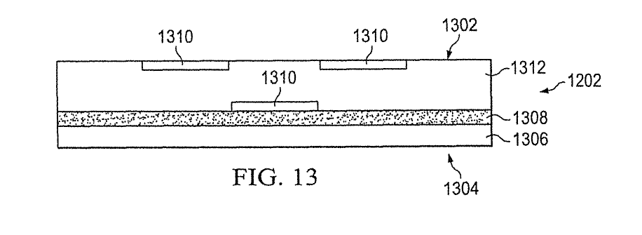

Referring now to FIG. 13, an end cutaway view of a portion of the plasma pouch 1200 is shown. The body portion 1202 can be seen to comprise an inner side 1302 corresponding to the interior 1210 of the pouch 1200, and an outer side 1304 corresponding to an exterior of the pouch 1200. The outer side 1304 may be covered by a flame and shock retardant material 1306 comprising an outer layer. This material 1306 may be similar to, or the same as, material utilized in fire resistant blankets. This may help to prevent any damage due to electricity or plasma to any objects or supporting surfaces outside the pouch 1200. The material 1306 may also protect against shorting or burnout of interior dielectric material.

A substrate 1308 may be provided under, or next to, the outer layer material 1306. The substrate 1308 may comprise materials such as Teflon® or polyethylene film. The substrate 1308 seals at least some of a plurality of electrodes 1310 against contact with air, and thus prevents generation of plasma on sealed surfaces. The pattern of the electrodes 1310 in the pouch can also implement various geometries (e.g., as discussed above). Thus, flow within the pouch 1200 can be controlled based on electrode geometry. In some embodiments, metallic tape or etched powdered electrodes may be used due to their flexibility.

The electrodes 1310 are restrained in a dielectric medium 1312. In some embodiments, the medium 1312 is a flexible film. This provides flexibility for the pouch 1200 and increases the number of geometries of electrodes that can be generated. The medium 1312 may range from less than 0.005 inches to about 0.010 inches in thickness. The thickness of the entire layer 1202 is only a few millimeters thick in some embodiments.

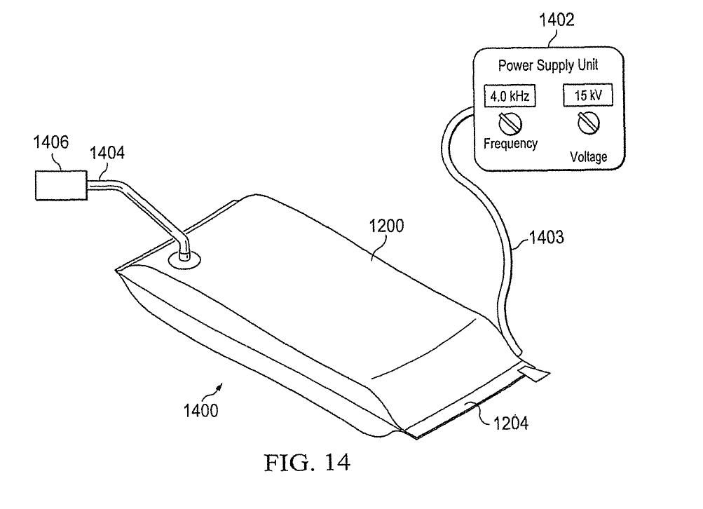

Referring now to FIG. 14, a perspective view of a system 1400 employing the plasma pouch 1200 of FIG. 12 for decontamination purposes is shown. The system 1400 employs a power supply 1402 that includes a transformer and a wall supply plugin. The power supply may provide a fixed voltage and frequency. In other embodiments, the power supply may have a variable voltage. In some cases the range will be from about 5 kV to 20 kV and may have a frequency between 600-5000 Hz. Switches and other controls may be provided for operation of the power supply 1402.

The power supply 1402 is electrically connected to the plasma pouch 1200 and to the internal electrodes (e.g., 1310 of FIG. 13). It is understood that a plurality of electrical leads may be combined into a single cord 1403 that enters the pouch 1200 (or pouch wall 1202) for connection to the electrodes 1310.

In operation, it may be useful to evacuate a certain amount of air from the pouch 1200 once the object to be decontaminated has been placed inside. This may result in a drop in the internal pressure of the pouch 1200 and/or a tendency for the pouch walls 1202 to adhere to the exterior of the contaminated object's surface. This helps reduce the distance between the plasma and the contaminated surface, allowing short lived species, such as Reactive Oxygen Species (ROS), to reach the surface of the object to be disinfected or sterilized.

The opening 1204 of the pouch 1200 may be sealable to prevent any gases and/or plasma generated species from escaping. This results in a more efficient inactivation. It also prevents a number of unwanted volatile gases and hazardous contaminants from escaping and potentially damaging nearby equipment or becoming a hazard to personnel.

Internally within the pouch 1200, vortices are generated due to the body forces in surface discharges. This results in better mixing of all the generated species to produce a very lethal “antimicrobial soup”. The products generated in the process (e.g., ozone), may be ventilated out through a filter unit 1406 attached to outlet hose 1404. Activated carbon is one filter media that may be used. Other reducing agent embedded filters may also reduce byproducts such as ozone to a less harmful form. In a similar fashion, a number of other materials can be used to adsorb other products such as ROS.

The pouch 1200 and/or the entire system 1400 may also be used for the purpose of cleaning surfaces through etching of both organic and inorganic molecules. Gaseous mixtures such as O₂ and CF₄ have a high etching ability when used as feed gas for plasma instead of air. In one embodiment, they are injected into the pouch 1200 via outlet hose 1404. Valving (not shown) may be utilized to allow the same hose 1404 to be used for evacuation of gases and by product and the introduction of gases into the pouch 1200.

The pouch 1200 may have a number of sensors and actuators to monitor its performance. For example, the pouch 1200 may contain proximity sensors and/or electric relays to shut down the discharge if a short or burn-out is detected. Ozone and other particulate concentration sensors may be used to detect leaks in pouch 1200.

In some embodiments, the pouch 1200 may incorporate the use of dyes or other reactive chemical agents. For example, an azo dye can be used to determine whether a required sterility level has been achieved. Based on laboratory results, the time frame utilized for sterilization may be adjusted.

It is understood that the pouch 1200 and/or the system 1300 can be replicated or expanded. For example, for large facilities, multiple pouch arrays can be established to run in tandem for large number of articles to be sterilized. It is also understood that multiple pouches 1200 may be operated by a single power supply 1402.

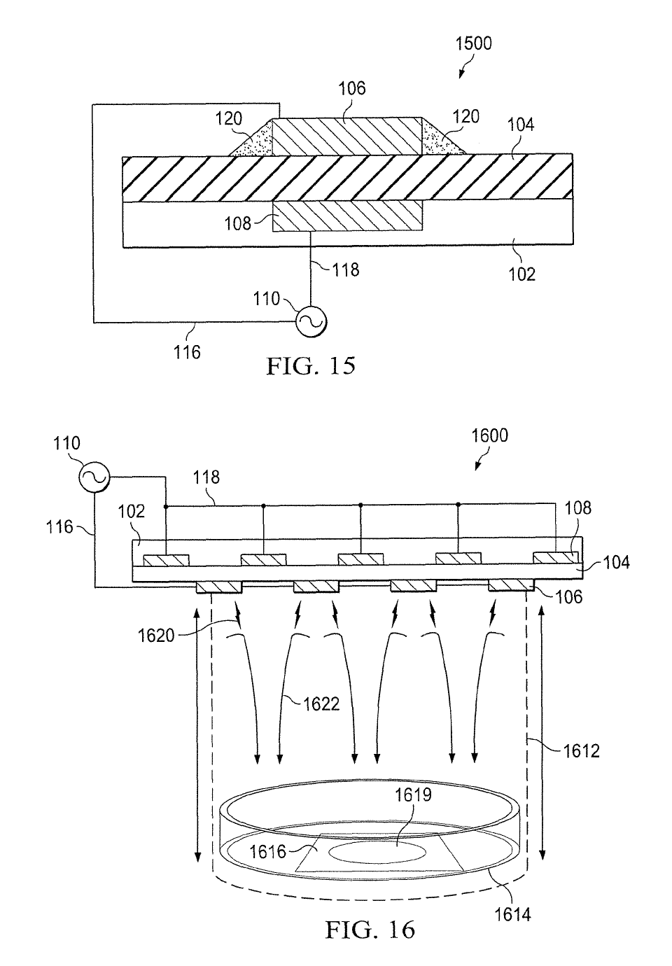

Referring now to FIG. 15, an embodiment of a surface dielectric barrier discharge (SDBD) plasma generating device with a symmetric electrode is shown. The device 1500 may be compared to the asymmetric device 100 of FIG. 1. Here, the device 1500 provides a substrate 102, which may be an insulator and/or part of an enclosure. A dielectric layer 104 interposes an exposed electrode 106 and an electrode 108 that is covered or sealed by the substrate 102. The electrode 106 attaches to a voltage source 110 by an electrical lead 116. The electrode 108 attaches to the voltage source 110 by an electrical lead 118. In the present embodiment, the voltage source 110 may include a power supply as well as any necessary transformers or circuit conditioning components to enable generation of plasma by application of sufficient voltage between the electrodes 106, 108 on the surface of the dielectric layer 104.

The device 1500 differs from the device 100 in the relative placement of the electrodes 106, 108. The device 1500, being a symmetric arrangement, has the electrode 106 centered, rather than offset, with respect to the electrode 108. Accordingly, two plasma regions 120 may be formed, one at each end of the electrode 106. Of course this configuration alters the motive forces produces by the plasma generating device 1500 compared to the device 100 of FIG. 100. Various embodiments of the present disclosure may be produced with either symmetric or asymmetric plasma generation configurations. However, in some embodiments, the asymmetric arrangement (e.g., the device 100 of FIG. 1) is preferred owing to an empirical determination that greater gas flow may be produced by the asymmetric design.

Referring now to FIG. 16, a schematic diagram of a system 1600 for cold plasma treatment of bacterial foodborne pathogens according to the present disclosure is shown. The system 1600 comprises a plasma generation device 1601 that is similar in many respects to that of FIG. 2. The plasma generation device 1601 comprises a plurality of exposed electrodes 106. A plurality of opposite electrodes 108 is separated from the exposed electrodes by a dielectric layer 104. The set of electrodes 108 opposite on the dielectric layer 104 from the electrodes 106 from which plasma is generated may be covered by an insulating layer 102. Both sets of electrodes 106, 108 may be affixed to a power supply 110 and operated as previously described to produce cold plasma from ambient air. The arrangement shown between the electrodes 106, 108 is an asymmetric arrangement, as discussed above. The system 1600 utilizes a cylindrical enclosure 1612 into which the plasma is discharged as shown by arrow 1620. A plasma flow is thereby produced as shown at arrows 1622 that directs the plasma to the contaminated subject.

The system 1600 of FIG. 16 was constructed and tested to prove the efficacy of systems and methods of the present disclosure. A petri dish 1614 was provided with a glass cover slip 1616 that was inoculated with contaminant bacteria 1618. A height “h” which separated the dish 1614 from the plasma generator 1601 was allowed to vary within the cylindrical enclosure 1612. This test was repeated with both symmetric and asymmetric electrode arrangements.

During testing of the system 1600, the dynamics of the induced airflow by the plasma generation device was evaluated by particle image velocimetry (PIV) and the efficacy in microbial inactivation was examined by using a five-strain cocktail of Listeria monocytogenes that was spot-inoculated onto the coverslip 1616 (which was otherwise sterile), placed at various distances (1, 3, 5, and 7 cm) from the plasma source, with inoculated untreated samples as controls.

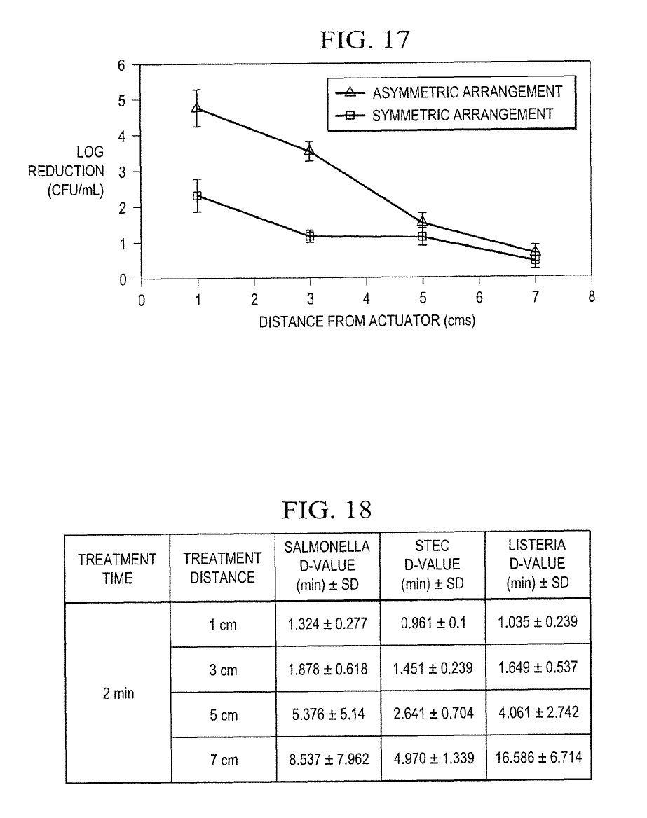

Bacterial inactivation was observed at all distances and treatment times but with decreasing efficiency at increasing distance. Shown in FIG. 17 are average log CFU/mL reductions for 4 min treatments log reductions of 4.8±0.5 vs 3.5±0.5 at 1 cm and 2.3±0.3 vs 1.1±0.2 at 3 cm for asymmetric and symmetric devices, respectively. The asymmetric arrangement of electrodes (106, 108, as shown) resulted in higher velocities and more turbulent flow than that of the symmetric arrangement. The PIV data was supported by microbial inactivation data, in which significant (p<0.05) higher log reduction of inoculated L. monocytogenes was achieved by the device with asymmetric arrangement of electrodes than that of symmetric ones.

Common bacterial foodborne pathogens were further shown experimentally to be inactivated by cold plasma treatment using the device 1600 and the pouch 1200 when inoculated onto both biotic and abiotic surfaces. Bacterial inactivation was evaluated on sterile glass coverslips, pecans, and cherry tomatoes that were spot inoculated with multiple-strain suspensions of Salmonella enterica (Se), Shiga toxin-producing Escherichia coli (STEC), or Listeria monocytogenes (Lm) (107 CFU (colony forming units)/sample), air dried, and treated with the cold plasma devices herein described for 2 and 4 min at 1, 3, 5, and 7 cm. Inactivation of bacterial cells was observed at all distances and at both treatment times but with decreasing efficiency at increasing distance and shorter treatment times. Average log CFU/mL reductions for 4 min treatments at 1 cm were 3.02 for Se, 3.61 for STEC, and 3.99 for Lm. D-values (min) at 1 cm were 1.32 for Se, 0.96 for STEC, and 1.04 for Lm. An approximately 1 and 2 log CFU/mL reduction was observed on pecans and cherry tomatoes at 4 and 10 min, respectively. Particle image velocimetry (PIV) was used to evaluate induced airflow dynamics and PIV data revealed that the electrode arrangement influences the induced localized airflow due to the coupling of the electric field into the neighboring fluid (air). These results confirmed that the cold plasma actuator design within the devices of the present disclosure induces a localized airflow that propels reactive species to distant surfaces. Additionally, SDBD can be used to successfully inactivate common bacterial pathogens with increased efficiency in close proximity to SDBD actuators.

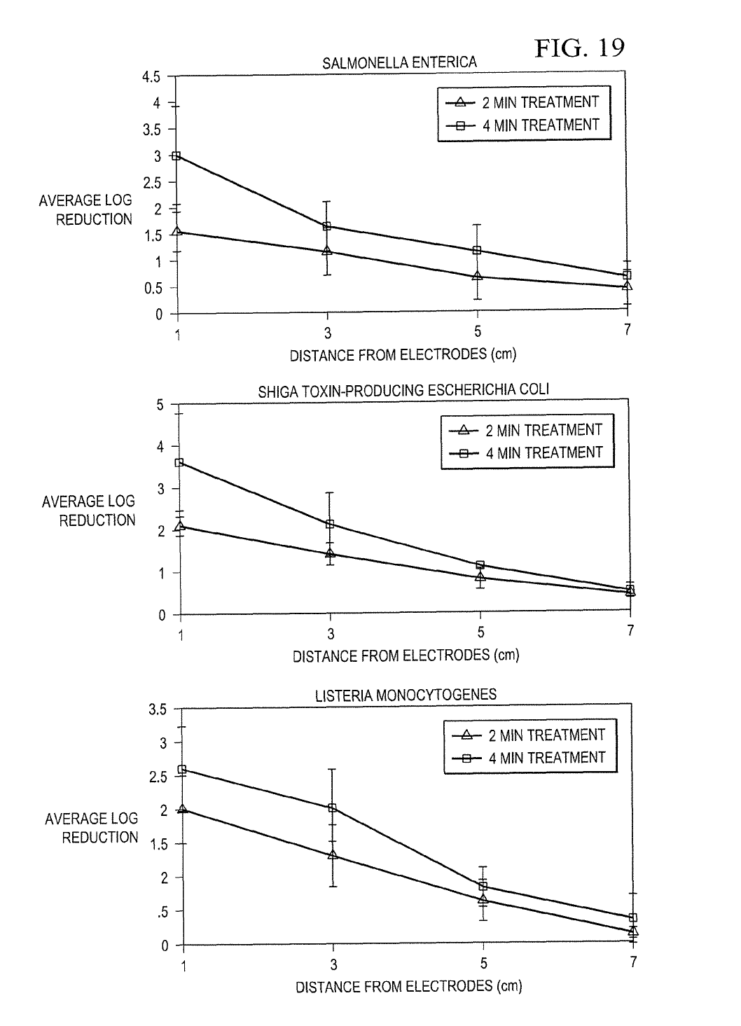

Full data for the instant experiment may be seen in FIG. 18. Plots of the results for each of the tested strains (average log reduction time versus distance from electrodes) are shown in FIG. 19. From the foregoing, it can be seen that the developed ambient-air, cold plasma systems and methods of the present disclosure can effectively inactivate at least three major foodborne bacterial pathogens.

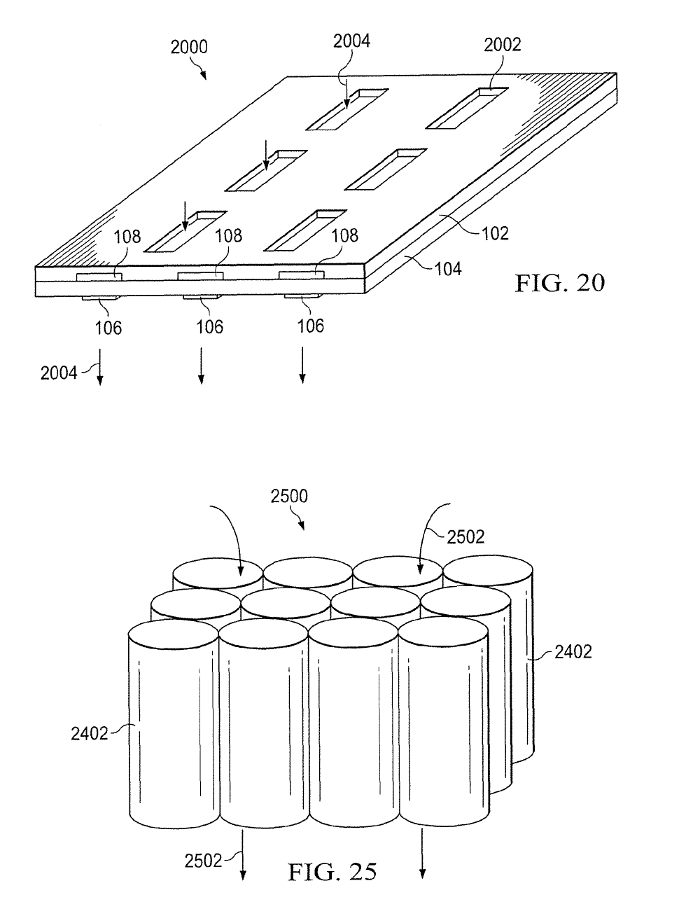

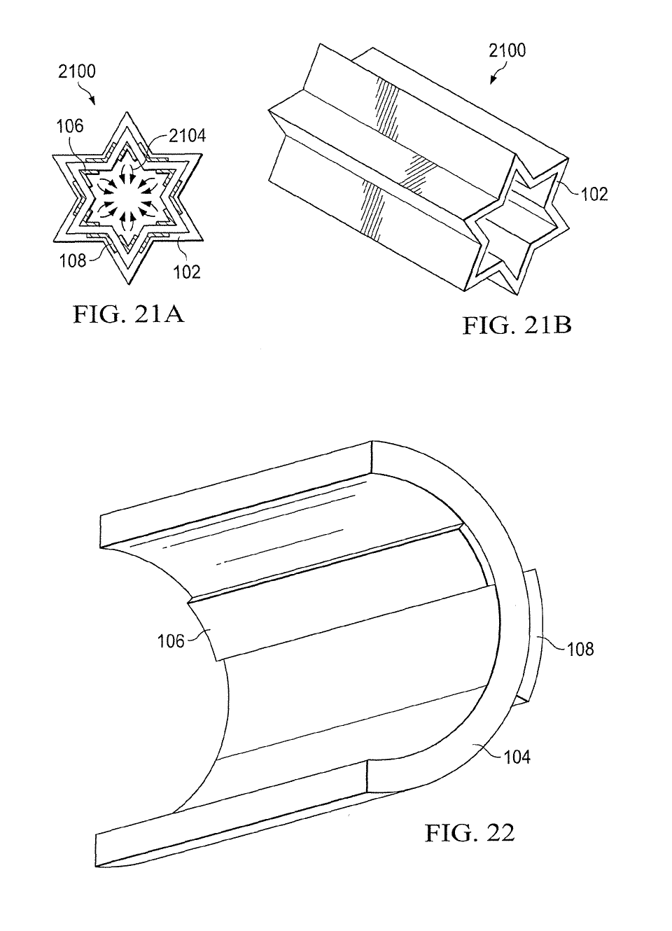

The experimental results above are intended to provide proof of efficacy of systems and method of the present disclosure. However, the systems may be physically adapted to operate in, or as a part of, a continuous process. FIGS. 21-25 elaborate on these concepts. FIG. 20 is a perspective view of a cold plasma system having a grid-like substrate. The insulator or substrate layer 102 is provided with grid-like perforations 2002. The perforations 2002 may pass completely through the structure 2000 including the dielectric layer 104. The electrodes 106, 108 may be positioned such that the induced plasma flow serves to draw air through the thickness of the device 2000 as shown by arrows 2004. For simplicity, the power supply 110 and leads 116, 118 (e.g, FIG. 1) are omitted.

The configuration of FIG. 20 allows for food substances that can be carried in air flow (e.g., powders, etc.) to be drawn through the perforations 2002 and encounter the generated cold plasma, which also serves to promote air flow through the perforations. The device 2000 could also be reversed such that the generated plasma tended to push against gravity by producing an upward thrust. This configuration would tend to suspend particles in plasma longer where such is needed.

Referring now to FIG. 21(a), a schematic diagram of a cold plasma system 2100 having a 3-dimensional chevron shape is shown. Here the insulator 102 forms the housing and seals electrodes 108. The dielectric layer 104 follows the general contour of the insulator 102 and supports electrodes 106 inside the housing 102. The induced flow will generally be toward the center of the device 2100 as shown by arrows 2104. Here again, the power supply and power leads are omitted for simplicity. FIG. 21(b) illustrates the three dimensional shape of the outside of the device 2100. Again, such a configuration may be useful in certain processes where contaminated foods are moved through the device 2100. The induced plasma flow will not only serve to bring plasma into contact with the contaminated food product, but will also tend to keep any food product from coming to rest against the inside of the device 2100.

Plasma systems may also be built around a cylindrical or semi-cylindrical configuration. FIG. 22 is a simplified schematic diagram of a semi-cylindrical electrode configuration. The dielectric 104 may be curved to form a semi cylinder. The electrodes 106, 108 may be symmetric or offset with respect to one another around the semi cylinder. An insulating layer, leads, and power supply (not shown) may be utilized to complete the system.

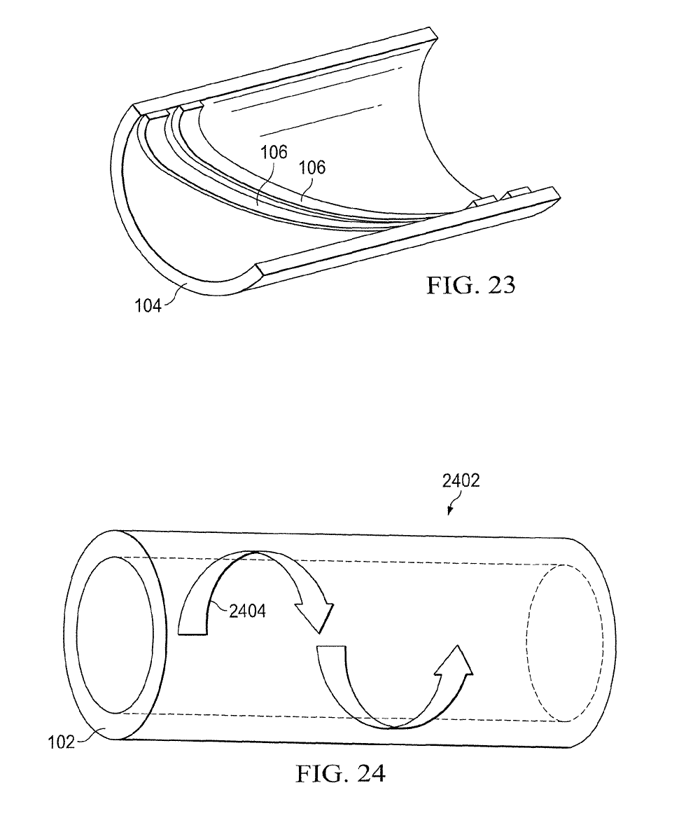

Referring now to FIG. 23, a simplified schematic cutaway diagram of a cylindrical electrode configuration is shown. Here, for simplicity, only the inner electrodes 106 and the dielectric 102 are shown for simplicity. The remaining components described above are assumed. The spiraling of the electrodes 106 may be employed to produce a flow within the cylinder, as seen, for example, in FIG. 24. Here the substrate 102 and the internal dielectric layer form a cylindrical system 2402. The sealed electrodes (not shown) follow (or slightly lead) the inner electrodes 106 in order to generate plasma that provides a motive force as shown by arrows 2402. This air (and contaminated food) are provided with a motive force from the cold, ambient-air plasma.

Referring now to FIG. 25, a perspective view of a cold plasma system built from an array of cylindrical electrodes is shown. The system 2500 employs a plurality of cylindrical devices 2402. These may be oriented to promote plasma (and air/gas) flow through the array as shown by arrows 2502. The cylinders 2402 could be configured to generate upward or downward thrust. Hence the system 2500 might work with or against gravity as the system 2000 of FIG. 20. It will be appreciated that either system (2000, 2500) could be oriented to provide lateral plasma flow as well.

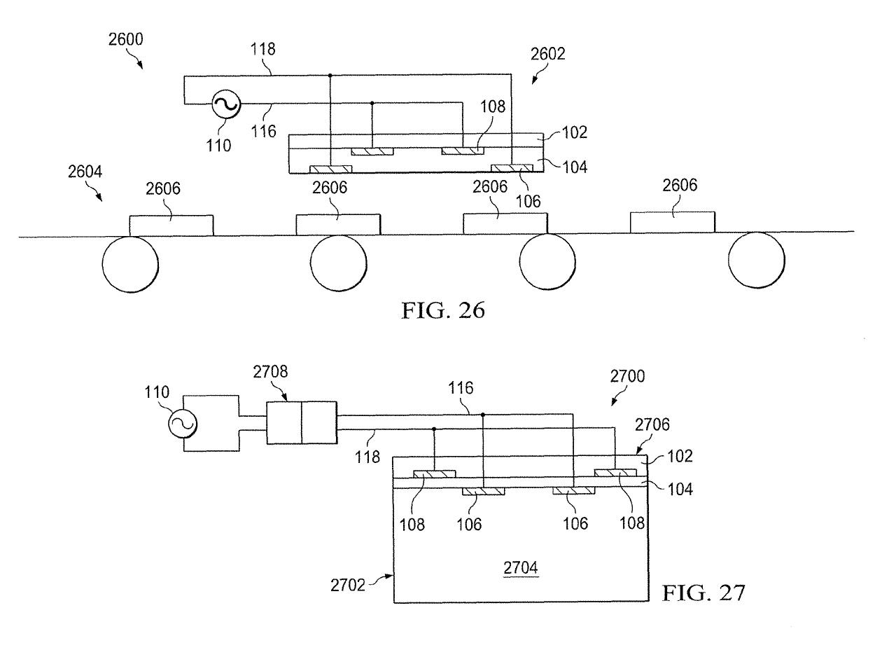

Various embodiments of the present disclosure may be readily adapted for use in existing shipping, storage, and processing mechanisms. For example, as shown in FIG. 26, a food processing system 2600 may include a conveyor system 2604, as are known in the art. In proximity to the conveyor system 2604 is a cold plasma system 2602 constructed according to the present disclosure. The cold plasma system 2602 may be continuously or selectively activated to produce plasma in proximity to food items 2606 passing by the cold plasma system 2602 on the conveyor system 2604. It will be appreciated that the operation of the conveyor system 2604 may be made to coincide with the operation of the cold plasma system 2602. For example, the conveyor system 2604 may be operated continuously or selectively to stop and allow food items 2606 adequate time for decontamination by plasma from the cold plasma system 2602. In other embodiments, the conveyor system 2604 may operate more slowly than is needed for full decontamination of the food items 2506. In such cases, the cold plasma system 2602 may be paused when not needed.

FIG. 27 illustrates a shipping or storage system 2700 employing the cold plasma generation techniques of the present disclosure. A container 2702, defining an interior space 2704, may be formed with all or a portion of a cold plasma generation system built into one or more of the container walls. Here, an upper container wall 2706 comprises the substrate 102, dielectric 104 and electrodes 106, 108, all arranged to generate cold plasma within the interior space 2704. Leads 116, 118 may pass out of the container and fit to the power supply 110 by a plug 2708. It should be understood that the plug 2708 might be flush fitted with the container wall 2706 or another container wall such that the power supply 110 may simply be electrically connected straight into the container 2700.

The configuration shown in FIG. 27 allows the power supply 110 to be reused as the container 2700 is discarded or recycled. The container 2700 may be a shipping or storage container. In some embodiments, the power supply 110 may be integrated with the container 2700 such that the contents of the container 2700 can be continuously or periodically decontaminated during the shipping process. Via the use of appropriate electronics, plasma generation may be made to occur on a period basis depending upon the contents of the container 2700.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method comprising:
arranging a first electrode and second electrode in an asymmetric relationship on opposite sides of a dielectric layer;
providing an insulating covering on the first electrode;
applying a power source to the first and second electrodes;
creating a voltage between the first electrode and the second electrode in ambient atmosphere to create a cold plasma; and
exposing a contaminated food product to the cold plasma.

2. The method of claim 1 further comprising arranging the insulating covering to create an enclosure.

3. The method of claim 2, wherein the enclosure comprises a cylinder.

4. The method of claim 3, wherein the first electrode and second electrode are arranged in the cylinder to promote the flow of plasma through the cylinder.

5. The method of claim 4, wherein the food product is a powder flowing through the cylinder.

6. The method of claim 1, further comprising configuring the dielectric and the insulated covering to form a grid defining a plurality of perforations therethrough.

7. The method of claim 6, further comprising arranging the first electrode and second electrode to promote flow of gases through the grid.

8. The method of claim 7, wherein the food product is a powder flowing through the grid.

9. The method of claim 1, further comprising placing the dielectric layer in proximity to a food carrying conveyor system for decontamination of food items in transit on the conveyor system.

10. The method of claim 1, further comprising forming the dielectric layer and the insulating covering into a portion of a container for decontamination of contents of the container.

11. A method comprising:
placing a substrate so as to define an at least a portion of an interior volume;
placing a dielectric layer on the substrate in the interior volume;
placing a plurality of electrodes immediately adjacent to the dielectric layer such that at least one electrode is exposed to the interior volume and at least one electrode is insulated by the substrate;
placing a food product into the interior volume in the presence of ambient atmospheric gases;
providing an excitation voltage between the electrodes to produce cold plasma directed to contact with the food product in the interior volume.

12. The method of claim 11, wherein the step pf placing a plurality of electrodes further comprises placing at least two electrodes in a symmetric relationship with respect to one another on opposite sides of the dielectric.

13. The method of claim 11, wherein the step pf placing a plurality of electrodes further comprises placing at least two electrodes in an offset relationship with respect to one another on opposite sides of the dielectric.

14. The method of claim 11, further comprising producing plasma for contact with the food product long enough to destroy food borne pathogens.

15. The method of claim 11, further comprising applying a voltage to electrodes on both sides of the dielectric layer.

16. The method of claim 11, further comprising arranging the substrate into a cylinder such that plasma is produced inside the cylinder.

17. The method of claim 16, further comprising arranging the substrate into multiple cylinders each with a plurality of electrodes such that plasma is produced inside each cylinder.

18. The method of claim 16, further comprising arranging at least one interior electrode in a spiral within the cylinder.

19. The method of claim 11, further comprising arranging the substrate into a three-dimensional chevron shape such that plasma is produced therein.
